(19) United States
(12) Patent Application Publication
(10) Pub. No.: US 2025/0249172 A1
(43) Pub. Date: Aug. 7, 2025

(54) PUMP WITH EVACUATED DRIVER FOR AUTO-INJECTION

(71) Applicant: AG IP HOLDING, LLC, Okemos, MI (US)

(72) Inventor: Patrick Kenneth Powell, Grosse Pointe Park, MI (US)

(21) Appl. No.: 18/854,712

(22) PCT Filed: Apr. 11, 2023
(86) PCT No.: PCT/US2023/018121

§ 371 Date: Oct. 7, 2024

Related U.S. Application Data
(60) Provisional application No. 63/329,627, filed on Apr. 11, 2022.
(60) Provisional application No. 63/342,653, filed on May 17, 2022.
(60) Provisional application No. 63/345,930, filed on May 26, 2022.
(60) Provisional application No. 63/358,334, filed on Jul. 5, 2022.
(60) Provisional application No. 63/358,337, filed on Jul. 5, 2022.
(60) Provisional application No. 63/359,262, filed on Jul. 8, 2022.
(60) Provisional application No. 63/388,405, filed on Jul. 12, 2022.
(60) Provisional application No. 63/389,205, filed on Jul. 14, 2022.
(60) Provisional application No. 63/389,496, filed on Jul. 15, 2022.
(60) Provisional application No. 63/405,173, filed on Sep. 9, 2022.
(60) Provisional application No. 63/416,647, filed on Oct. 17, 2022.
(60) Provisional application No. 63/482,116, filed on Jan. 30, 2023.

(51) Int. Cl. A61M 5/152; A61M 5/142; A61M 5/145

(52) U.S. Cl. CPC A61M 5/152; A61M 5/14248; A61M 2005/14256; A61M 2005/14506; A61M 2205/364; A61M 2207/00; A61M 2209/045

(57) ABSTRACT

A pump includes a container that has an interior container volume for holding a substance. There is an outlet associated with the interior container volume for discharging the substance. A hollow needle is connected with the outlet. There is a substance driver disposed in the interior container volume. The substance driver has a stored potential energy that is releasable as kinetic energy and, upon release, the substance driver expands against the substance in the interior container volume and thereby discharges the substance through the outlet to the hollow needle.

### BACKGROUND

Large volume drug injection, such as intravenous (IV) therapy, can be expensive and difficult for some people to access if not covered by insurance. Moreover, clinics for IV therapy may not be available or may be located far away, making treatment especially difficult for the elderly or people with mobility issues. Even if available, IV therapy can take several hours per treatment and can be uncomfortable, particularly if the needle is not inserted correctly or if the medication being administered is irritating to the veins or tissues.

A wearable, large volume, drug delivery device would enable patients to self-administer therapy in a home setting. Self-administration would allow people to take medication at a time and place that is convenient for them, without having to make appointments or travel to a healthcare facility. Additionally, the therapy is conducted in private, without having to discuss medical conditions or medication use with others, and self-administration would allow substantial control over medication use and decision-making about treatment. Present IV therapies, however, have posed challenges to adaptation as a wearable, large volume drug delivery for self-administration.

### SUMMARY

A pump according to an example of the present disclosure includes a container that has an interior container volume for holding a substance, an outlet associated with the interior container volume for discharging the substance, a hollow needle connected with the outlet, and a substance driver disposed in the interior container volume. The substance driver has a stored potential energy that is releasable as kinetic energy and, upon release, the substance driver expands against the substance in the interior container volume and thereby discharging the substance through the outlet to the hollow needle.

In a further embodiment of any of the foregoing embodiments, the outlet includes a conduit connecting the interior container volume with the hollow needle.

A further embodiment of any of the foregoing embodiments includes a needle container in which the hollow needle is disposed and a needle driver disposed in the needle container adjacent the hollow needle, the needle driver has a stored potential energy that is releasable as kinetic energy and, upon release, the needle driver expands and thereby drives the hollow needle to protrude from the needle container.

In a further embodiment of any of the foregoing embodiments, release of the substance driver causes the substance to flow through the outlet to the hollow needle, and the pressure of the flowing substance moves an element to release the needle driver.

In a further embodiment of any of the foregoing embodiments, the substance driver includes a casing and a spring device in the casing. The casing is evacuated to define a pressure differential that stresses the spring device to provide the stored potential energy, a pressure equalization port that is operable to release the pressure differential and, upon release, the spring device expands to increase the volume of the casing and thereby generate a vacuum in the casing.

In a further embodiment of any of the foregoing embodiments, the pressure equalization port is connected by an inlet conduit to an inlet at the needle container, and the inlet is covered by a seal that is removeable to thereby release the pressure differential via the inlet conduit.

A further embodiment of any of the foregoing embodiments includes a needle cover on the needle container and a needle cover driver coupled with the needle cover. The needle cover driver has a stored potential energy that is releasable as kinetic energy and, upon release, the needle cover driver expanding and thereby moving the collapsible needle cover from store position to a deployed position shielding the hollow needle.

In a further embodiment of any of the foregoing embodiments, the driving of the needle to protrude from the needle container causes the needle to pierce the needle cover driver and thereby release the needle cover driver.

In a further embodiment of any of the foregoing embodiments, the driving of the needle to protrude from the needle container causes the needle to pierce the needle cover driver and release a flowable substance from inside the needle cover driver to the needle puncture site.

A further embodiment of any of the foregoing embodiments includes a base has opposed first and second sides, at least the container is attached on the first side, and the second side includes an adhesive.

In a further embodiment of any of the foregoing embodiments, the container includes at least one flexible bladder with a fill port for holding the substance.

In a further embodiment of any of the foregoing embodiments, the substance driver includes a casing and a constant force spring in the casing. The casing is evacuated to define a pressure differential that holds the constant force spring in a wound state around an axis to provide the stored potential energy, a pressure equalization port that is operable to release the pressure differential and, upon release, the constant force spring rotates to increase the volume of the casing to expand against the substance.

A further embodiment of any of the foregoing embodiments includes an attachment member configured to affix the pump to an object.

In a further embodiment of any of the foregoing embodiments, the substance driver includes a casing and a spring device in the casing. The casing is evacuated to define a pressure differential that stresses the spring device to provide the stored potential energy, a pressure equalization port that is operable to release the pressure differential, and further including a vessel that has a pressurized chamber in which at least the container and the substance driver are disposed. The pressure of the pressurized chamber is greater than a pressure in the casing of the substance driver in order to prevent premature release of the substance driver.

A further embodiment of any of the foregoing embodiments includes a heater module attached with the vessel.

In a further embodiment of any of the foregoing embodiments, the heater module includes a heater module container, a thermal material in the heater module container that is exothermically reactive with air to produce heat, and a heater module driver disposed in the heater module container. The heater module driver has a stored potential energy that is releasable as kinetic energy and, upon release, the heater module driver expanding and drawing in air through at least one air inlet to the thermal material and produce heat.

A method for assembling a pump according to an example of the present disclosure includes providing a container that has an interior volume and an outlet associated with the interior volume, introducing a medicament into the interior volume, and installing a substance driver at least partially into the interior volume of the container. The substance driver has a stored potential energy that is releasable as kinetic energy and, upon release, the substance driver is expandable against the medicament in the interior volume to thereby discharge the substance through the outlet.

A further embodiment of any of the foregoing embodiments includes evacuating an interior of the substance driver prior to installing the substance driver at least partially into the interior volume of the container.

A method of treatment according to an example of the present disclosure includes placing a pump as in any of the foregoing embodiments on a patient, connecting the outlet of the pump to the patient, and releasing the substance driver. The substance driver expands against the medicament in the interior container volume and thereby discharges the medicament through the outlet and into the patient. After discharge of the medicament into the patient, the pump is removed from the patient.

In a further embodiment of any of the foregoing embodiments, the connecting includes penetrating a hollow needle into the patient.

A further embodiment of any of the foregoing embodiments further includes a needle cover on the needle container and a needle cover driver coupled with the needle cover. The needle cover driver includes a casing and a spring device in the casing. The casing is expandable/collapsible and is evacuated to define a pressure differential across the spring device, relative to the surrounding ambient environment pressure. The vacuum in the casing causes the stressing of the spring device to provide stored potential energy. The spring device can be stressed under the vacuum force of the collapsed casing to store potential energy and then elastically recover to release the potential energy once the pressure differential is equalized. The needle cover may also be used on other devices independently of any of the foregoing embodiments.

A method for use with a pump according to an example of the present disclosure includes attaching a pump as in any of the foregoing embodiments to a patient, and releasing the substance driver, the substance driver expanding against the medicament in the interior container volume and thereby discharging the medicament through the outlet and into the patient.

The present disclosure may include any one or more of the individual features disclosed above and/or below alone or in any combination thereof.

### BRIEF DESCRIPTION OF THE DRAWINGS

The various features and advantages of the present disclosure will become apparent to those skilled in the art from the following detailed description. The drawings that accompany the detailed description can be briefly described as follows.

FIG. 1A illustrates an example pump for delivery of a fluid.

FIG. 1B illustrates the pump of FIG. 1A during delivery.

FIG. 2 illustrates another example pump with a needle extender.

FIG. 3 illustrates the pump of FIG. 2 upon release of the driver.

FIG. 4 illustrates the pump of FIG. 2 during delivery.

FIG. 5 illustrates another example pump with a deployable needle cover.

FIG. 6A illustrates the pump with the needle cover deployed.

FIG. 6B illustrates a needle cover that contains a fluid agent.

FIG. 6C illustrates the needle cover after a needle passes through the cover and the fluid agent enters the puncture site.

FIG. 7 illustrates another example pump with an inlet on the needle extender that releases the substance driver.

FIG. 8 illustrates the pump of FIG. 7 during delivery.

FIG. 9 illustrates another example pump that includes a flexible bladder.

FIG. 10 illustrates the pump of FIG. 9 during delivery.

FIG. 11 illustrates another example substance driver that may be used with an intravenous fluid bag.

FIG. 12 illustrates another example substance driver that may be used with an intravenous fluid bag.

FIGS. 13 and 14 illustrate an example implementation in which there are multiple pumps.

FIG. 15 illustrates an example in which the pump is sealed in a vessel to prevent premature release.

FIG. 16 illustrates an example in which the pump is sealed in a vessel that has a heater module.

FIG. 17 illustrates the pump of FIG. 15 upon removal from the vessel.

In this disclosure, like reference numerals designate like elements where appropriate and reference numerals with the addition of one-hundred or multiples thereof designate modified elements that are understood to incorporate the same features and benefits of the corresponding elements.

### DETAILED DESCRIPTION

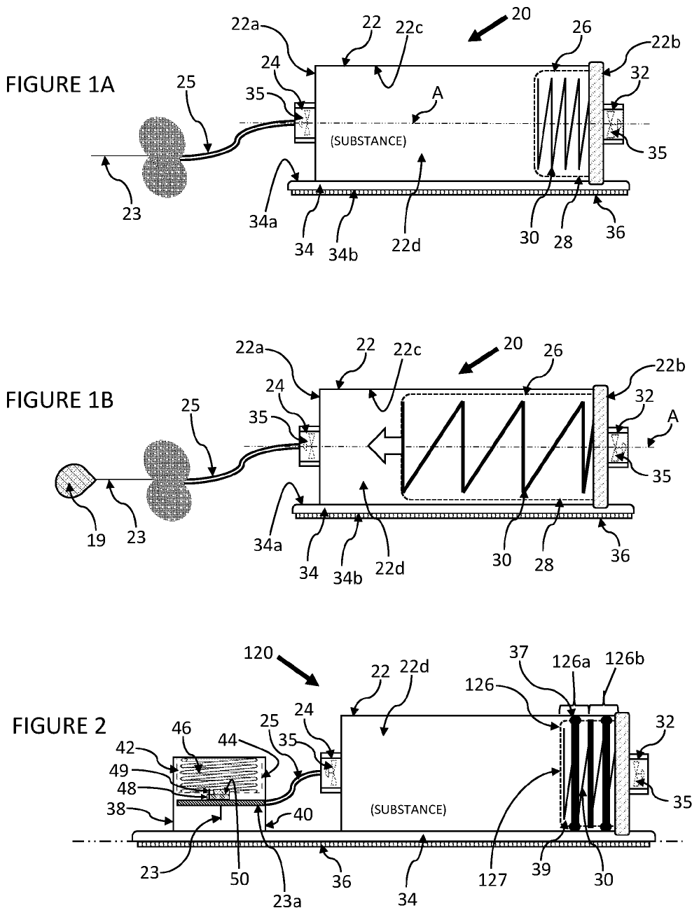

FIG. 1A schematically illustrates a pump 20. The pump 20 may be used for delivery of a fluid, such as but not limited to, a liquid, a gas, a semi-solid, a gel, a suspension, a flowable powder, a flowable solid (e.g., solid that transitions to liquid form at elevated conditions), or combinations of these. A contemplated implementation of the pump 20 is in the medical industry to deliver a large amount of medication subcutaneously (under the skin) over an extended period of time, although the pump 20 is not limited to such a use and may be used in other types of end-uses to inject fluids. In some instances, the substance may be initially solid (as with a frozen vaccine or drug suspended in wax) and then softened for delivery, or the substance may be a mixture of other substances, such as a reconstitution with a fluid and a powder (e.g., a lyophilized pharmaceutical).

The pump 20 includes a container 22. In this example, the container 22 defines a central axis A and has first and second end walls 22a/22b and a side wall or walls 22c. The walls 22a/22b/22c define an interior container volume 22d for holding a substance (fluid) that is to be pumped. As an example, the container 22 is sized for large volume medicament injection over an extended time period or in several time intervals. In this regard, the pump 20 may be incorporated into a wearable that is designed to be worn on the body of a patient while the medicament is delivered. It is to be understood, however, that the size of the container 22 is not particularly limited and may be scaled up or down for a particular implementation.

In the illustrated example, the container 22 is cylindrical and is formed of a plastic material, such as but not limited to, polypropylene (PP), polyethylene (PE), polyvinyl chloride (PVC), cyclic olefin copolymers (COC) or cyclic olefin polymers (COP). However, the geometry and material of the container 22 can be varied. For example, the container 22 may alternatively be made of metal, glass, ceramic, elastomer, or combinations of these. Non-limiting examples may include stainless steel, aluminum alloys, borosilicate or soda lime glass, aluminum oxide, zirconium oxide, silicon carbide, hydroxyapatite, or silicon nitride, and natural or synthetic rubber. The container 22 may be opaque, translucent, or transparent to visible light, or a combination of these, and may be shaped symmetrical or asymmetrical. The container 22 may be of a multilayer construction, for example to prevent drug, oxygen, light or moisture penetration through the container walls in a complex shape. The medicament or other substance may be pre-loaded in the container 22 such that the pump 20 is ready for use, but the substance may alternatively be loaded into the container on-demand in preparation of the pump 20 for an imminent use.

The pump 20 includes at least one outlet 24 that is connected with the interior container volume 22d for discharging the substance. In the illustrated example, there is one outlet 24 and it is located at the first end wall 22a. It is to be appreciated, however, that there could be additional outlets, such as two outlets, three outlets, or more than three outlets, and the outlet or outlets may be located elsewhere on the container 22. In the illustrated example, the outlet 24 includes a flexible conduit 25, such as a tube, and there is a hollow needle 23 that is connected with the flexible conduit 25.

The pump 20 further includes a substance driver 26 at least partly disposed in the interior container volume 22d. The substance driver 26 occupies a region of the container 22, and the region is fluidly isolated from (i.e. sealed from) the interior container volume 22d. The substance driver 26 has a stored potential energy that is releasable as kinetic energy. Upon release, as shown in FIG. 1B, the substance driver 26 expands along a linear axial movement direction in the container 22 to take up at least a portion of the interior container volume 22d. In doing so, the substance driver 26 applies pressure against the substance in the interior container volume 22d and thereby causes the substance to discharge through the outlet 24 to the conduit 25 and hollow needle 23 (discharge shown as 19), where it is injected into a patient or other object.

The driver 26 in this example includes a casing 28 and a spring device 30 in the casing 28. The casing 28 is expandable and collapsible. In this regard, the casing 28 may include, but is not limited to, an elastically flexible sack, a convoluted sack, or other mechanical structure that permits the casing to expand/collapse. The interior of the casing 28 is evacuated and thus defines a pressure differential across the spring device 30, relative to the surrounding ambient environment pressure. That is, the vacuum in the casing 28 relative to the surrounding ambient pressure causes the casing 28 to collapse around, and thus stress, the spring device 30 to provide the stored potential energy. In general, the vacuum pressure in the casing 28 may be a low as <10⁻³ Pa but may be scaled up or down for a particular implementation.

The spring device 30 in the illustrated example is a coil spring. Alternatively, the spring device 30 may include, but is not limited to, a compressible foam, a compressible elastic, a compressible textile, a compressible fluid, a collapsible lattice structure, a pneumatic strut, a torsional spring, a constant force spring (e.g., clock spring) or combinations of different types of these or other types of springs, as long as the spring device 30 can be stressed under the vacuum force of the collapsed casing 28 and to store potential energy and then elastically recover to release the potential energy once the pressure differential is equalized. In some instances, the spring device 30 may compress when stressed (e.g., a coil spring or foam), but in other instances the spring device 30 may twist or otherwise deform when stressed (e.g., torsion spring or a constant force spring).

The vacuum in the casing 28 holds the spring device 30 in its stressed, elevated potential energy state (relative to the spring device 30 at rest) until the pressure differential is released. The pressure differential is released by opening the casing 28 to the surrounding ambient environment. Once opened, air or other substances from the surrounding environment enters into the casing 28, thereby equalizing the initial vacuum in the casing 28 with the ambient surroundings. Once equalized, without the vacuum holding the casing 28 in its collapsed state, the potential energy of the spring device 30 is converted to kinetic energy. Under the kinetic energy of the spring device 30, the casing 28 expands to take up at least a portion of the interior container volume 22d. The expansion of the casing 28 increases the interior volume of the casing 28, which generates a secondary vacuum in the casing 28 that draws an inflow of air or other substances from the surrounding environment into the casing 28 to equalize the secondary vacuum. The inflow to equalize this secondary vacuum may be controlled to thereby control the expansion of the casing 28 and concomitant discharge of the substance from the pump 20, as the discharge of the substance is proportional to the release of the secondary vacuum.

The casing 28 may be opened to release the pressure differential via an inlet port 32. The inlet port 32 extends thorough the second end wall 22b and connects the interior of the casing 28 to the surrounding ambient environment. The inlet port 32 is initially sealed, thereby preserving the vacuum in the casing 28 so that the potential energy is stored until released to activate the pump 20. The release can be triggered manually or in an automated fashion by opening the inlet port 32, such as by breaking a seal associated with the inlet port 32, opening a valve associated with the inlet port 32, or by operating or activating a regulator associated with the inlet port 32.

As also shown in the example of FIGS. 1A/1B, the pump 20 may include a regulator 35 for controlling the inflow to equalize the vacuum. The flow restriction serves to control expansion of the driver 26, which in turn controls (at least in part) the discharge rate of the substance from the outlet 24. For example, relatively low restriction of the inflow results in a rapid pressure equalization, rapid expansion of the driver 26, and a concomitant high rate of discharge. Inversely, relatively high restriction of the inflow results in a slower, gradual pressure equalization, slower expansion of the driver 26, and a concomitant low rate of discharge. Thus, the regulator 35 may be tailored to control the rate of inflow and thereby control the discharge rate of the substance. The initial vacuum pressure in the driver 26, characteristics of the spring device 30, volume of the casing 28, and size of the outlet 24 may also be selected in cooperation with the regulator 35 to modulate the discharge rate.

The regulator 35 may be selected from an orifice, a flow restriction tube, a fixed volume pump, an open cell foam, a valve, an electrical element, a viscous fluid, a mechanical clock valve, a solenoid, a wax, a thermally degradable material, a gravity vector sensitive port, or combinations thereof. A regulator 35 such as a flow restriction tube or orifice operate passively, while other types of regulators, such as a solenoid or electrical element, may be operated with an active control to modulate flow restriction and thus discharge.

Optionally, as shown in the illustrated example, the pump 20 is adapted to be affixed to an object, such as the body of a patient. In this regard, a base 34 is provided that has opposed first and second sides 34a/34b. The base 34 may contain at least a portion of a flat, rigid or semi-flexible substrate suitable for holding the pump 20 in an assembled state. The container 22 is attached on the first side 34a and the second side 34b includes an adhesive 36 for affixing the assembly to the object.

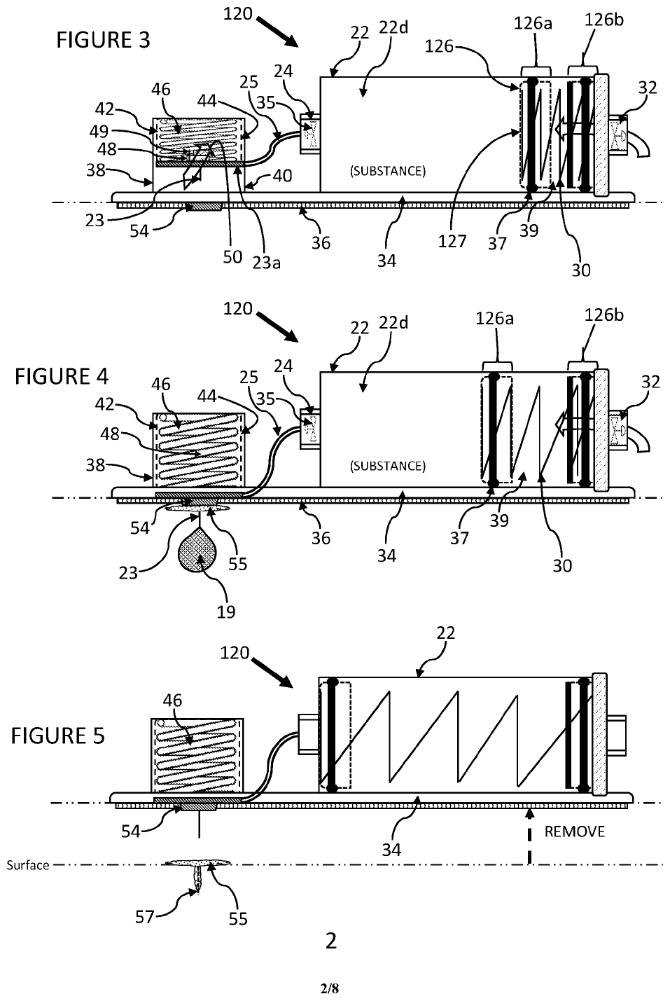

FIG. 2 illustrates a further example of a pump 120 but with substance driver 126 and a needle extender 38 that is configured to extend the needle 23 into the patient. The driver 126 includes two segments 126a/126b. The first segment 126a is linearly translatable in the container 22 (in the right-to-left direction in the figure), while the second segment 126b is fixed to the container 22. In this example, the driver 126 excludes the afore-described casing 28, although the casing 28 could be used. Instead, the first segment 126a includes a seal 37 that dynamically seals against the interior surface of the wall 22c of the container 22. The seal 37 thus isolates a region 39 from the remainder of the interior container volume 22d and thus functions similarly to the casing 28 in that regard. The spring device 30 is located in the region 39, which is evacuated. The resulting pressure differential holds the first segment 126a in a retracted position in which it compresses the spring device 30 to store the potential energy. Upon release of the pressure differential as shown in FIGS. 3-5, the spring device 30 is permitted to expand, pushing the first segment 126a against the substance (to the left in the figure) and thereby discharging the substance through the outlet 24. Optionally, the first segment 126a may include an inert face 127 in contact with the substance. The inert face 127 is unreactive with respect to the substance. For example, the inert face is a silica glass. The inert face 127 reduces contact between the substance and first segment 126a and seal 37, both of which may be made of an elastomer.

The needle injector 38 includes a needle container 40 in which the hollow needle 23 is initially disposed in a fully retracted position. There is a needle driver 42 adjacent the hollow needle 23, which may be attached to a pressure plate 23a for the needle driver 42 to press against to move the needle 23. The needle driver 42 is constructed similar to the substance driver 26 in that it includes a casing 44, a spring device 46 in the casing 44, and an inlet port 48, although the needle driver 42 could alternatively be of a segmented construction like the driver 126. Like the casing 28, the casing 44 is expandable/collapsible and is evacuated to define a pressure differential across the spring device 46, relative to the surrounding ambient environment pressure. The vacuum in the casing 44 stresses the spring device 46 to provide stored potential energy. The spring device 46 is a coil spring but alternatively may be a compressible foam, a compressible elastic, a compressible textile, a compressible fluid, a collapsible lattice structure, a pneumatic strut, or combinations of different types of these or other types of springs, as long as the spring device 46 can be stressed under the vacuum force of the collapsed casing 44 and to store potential energy and then elastically recover to release the potential energy once the pressure differential is equalized.

Upon release of the stored potential energy as kinetic energy, the needle driver 42 expands and thereby drives the hollow needle 23 to protrude from the needle container 40. In this example, the pump 20 is configured such that the release of the substance driver 26/126 triggers the near-simultaneous release of the needle driver 42. For example, the release of the substance driver 26/126 causes the substance in the container 22 to flow through the regulator 35 and flexible conduit 25 to the hollow needle 23 purging air from conduit 25 and hollow needle 23. As shown also in FIG. 3, the inlet port 48 includes a moveable element and there is a valve 50 associated with the moveable element and the flexible conduit 25 such that the pressurization of the substance in the conduit 25 causes the moveable element to press open valve 50 and thereby release the needle driver 42. In another embodiment, the moveable element is an expanding element including a breaker (sharp) 49 that pierces the casing 44 on expansion, releasing the driver 42 to expand. Upon release as shown in FIG. 4, the needle driver 42 urges the needle 23 via the pressure plate 23a to move and protrude from the needle container 40. Thus, when the needle injector 38 is against the skin of a patient, the needle 23 penetrates into the skin for injection of the medicament. Additionally, the stroke of the spring device 46 to penetrate the needle 23 into the patient may be less than the full available stroke of the spring device 46 to its full rest state. In this regard, upon penetration, the spring device 46 may serve to continue to apply pressure at the injection site via the pressure plate 23a. Such applied pressure during injection may facilitate pain reduction and enable continuous drug delivery during contortions of the skin surface.

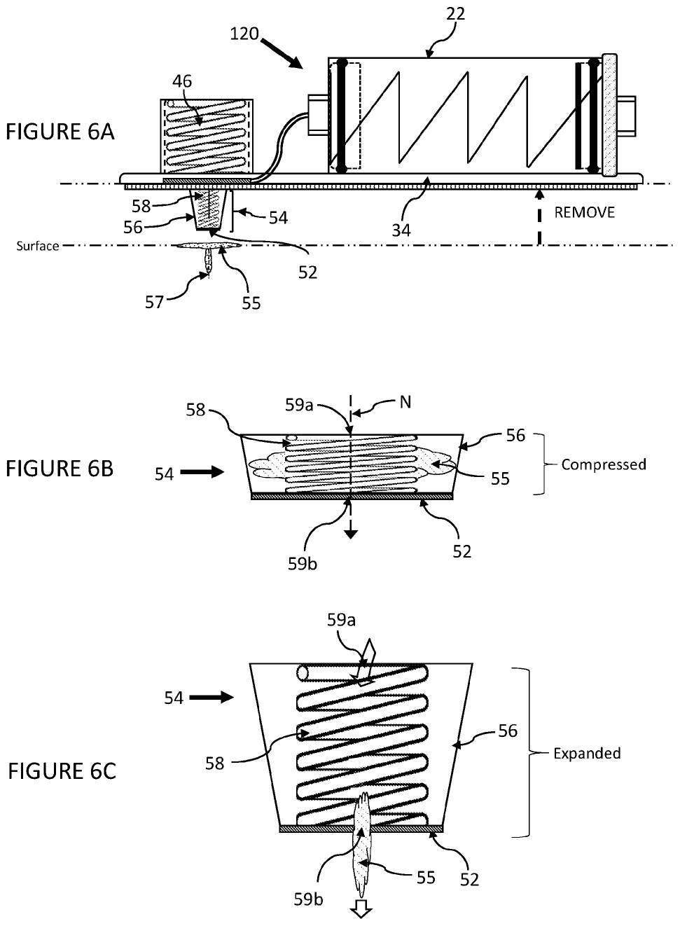

FIGS. 5, 6A, 6B, and 6C illustrate a further example of the pump 20 but with a needle cover 52 near the needle container 40, and a needle cover driver 54 coupled with the needle cover 52. The needle cover driver 54 is constructed similar to the substance driver 26 in that it includes a casing 56 and a spring device 58 in the casing 56. Like the casing 28, the casing 56 is expandable/collapsible and is evacuated to define a pressure differential across the spring device 58, relative to the surrounding ambient environment pressure. The vacuum in the casing 56 stresses the spring device 58 to provide stored potential energy. The spring device 58 is a coil spring but alternatively may be any of the aforementioned springs, as long as the spring device 58 can be stressed under the vacuum force of the collapsed casing 56 to store potential energy and then elastically recover to release the potential energy once the pressure differential is equalized.

Unlike the needle driver 42 and the substance driver 26, the needle cover driver 54 does not have an inlet port per se. Rather, upon release of the needle driver 42 to extend the needle 23 from the needle container 40, the needle 23 pierces the needle cover driver 54. The piercing creates puncture ports to release the needle cover driver 54 and equalize the pressure. Upon initial release, however, the spring device 58 is substantially prevented from expanding because the needle cover driver 54 is pressed against the skin of the patient. As shown in FIG. 6A, once the needle 23 and needle injector 38 are removed from the patient, the spring device 58 can then expand and thereby move the needle cover 52 to a deployed position in which it shields the needle 23, to reduce risk of inadvertent poking. Thus, during use of the pump 20, the needle 23 is only exposed when penetrating the skin, as it is in the needle container 40 prior to deployment and is shielded by the needle cover 52 upon retraction of the needle 23 from the skin.

In a further example of the needle cover 52 shown in FIG. 6B, the needle cover 52 contains a fluid agent 55, such as a medicament, sterility agent, an anesthetic, aloe vera, corticosteroids, antibiotics, antivirus agent, antifungal agents, sterility aid, alcohol, anesthetic agent, lidocaine, benzocaine, prilocaine, or tetracaine, sensation agent (hot or cold), menthol, camphor, capsaicin, allyl isothiocyanate, methyl salicylate, anti-allergic, antihistamines, decongestants, corticosteroids, leukotriene receptor antagonists, mast cell stabilizers, epinephrine (adrenaline), wound sealant, cyanoacrylates, or fibrin clotting, held under pressure differential until penetration of the needle 23 at 59a and 59b. The fluid agent 55 may coat the needle 23 as the needle 23 passes (along axis N) through the cover 54, after which the fluid agent 55 enters the puncture site, as shown in FIG. 6C. Additionally, the fluid agent 55, may spread around the puncture site, for affecting treatment, protection or comfort to the patient during the needle injection process. The spring device 46 when released may aid in expungement of the fluid agent 55 by applying a secondary pressure on the needle cover 54. The needle cover 54 may include a wicking feature, such as a cotton pad, to promote distribution of the fluid agent 55 around the puncture site. A residual amount of the fluid agent 55 may remain at the puncture site 57 after removal of the pump 120 from the body.

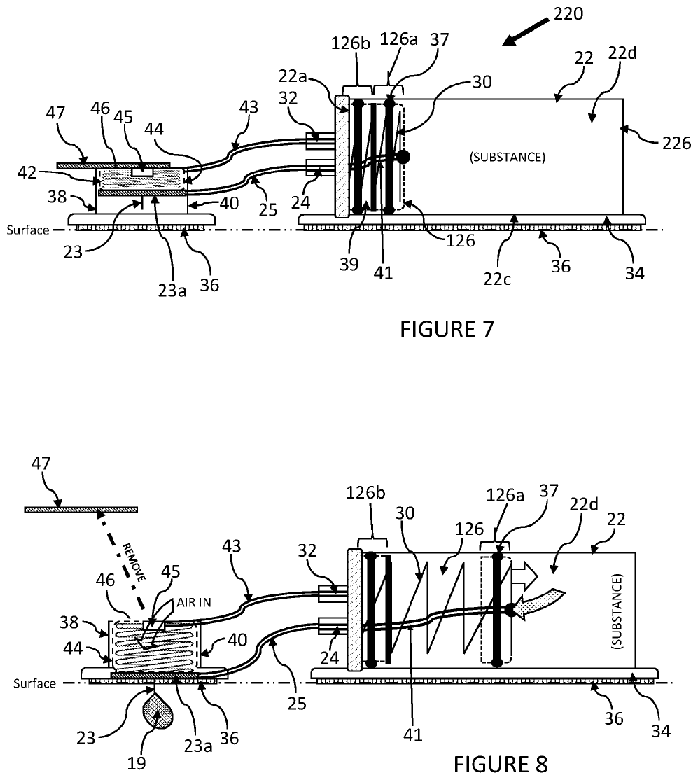

FIG. 7 illustrates another example pump 220 in which the first segment 126a of the driver 126 is linearly translatable in the container 22 (in the left-to-right direction in the figure), while the second segment 126b is fixed on the container 22. In this example, the inlet port 32 and the outlet 24 are both located at the first end wall 22a, which in this case may be a cap on the container 22. The outlet 24 is connected with the interior container volume 22d via a conduit 41 that extends through the driver 126. The inlet port 32 of the driver 126 is connected via a conduit 43 to an opening 45 on the container 40 of the needle injector 38. The opening 45 is initially covered with a seal 47. Upon removal of the seal 47 by a user, as shown in FIG. 8, the pressure differential in the substance driver 126 is released. The spring device 30 is permitted to expand, pushing the first segment 126a against the substance (to the right in the figure) and thereby discharging the substance through the conduit 41 then through the outlet 24 and through the conduit 25 to the needle 23. The opening 45 also serves as an inlet port for the needle driver 42. Thus upon removal of the seal 47, the needle driver 42 is released simultaneously (with the driver 126) to extend the needle 23.

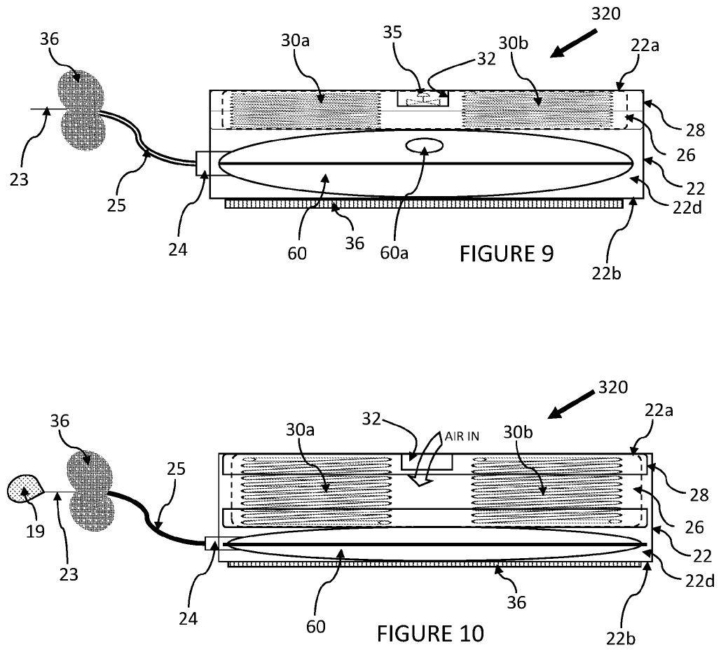

FIG. 9 illustrates another example of a pump 320. In this example, the pump 320 is oriented vertically such that the driver 26 is located near the top (first side 22a) of the container 22. A flexible bladder 60 is disposed in the interior container volume 22d. The flexible bladder 60 holds the substance and is connected to the outlet 24. The flexible bladder 60 may include a fill port 60a for depositing a substance or substances in the flexible bladder 60 or refilling the flexible bladder 60 with the substance for a re-use. Upon release of the driver 26, as shown in FIG. 10, the driver 26 expands against the flexible bladder 60, causing the substance to flow out of the flexible bladder 60 and through the outlet 24 to the conduit 25 and needle 23. As the cross-sectional area that the driver 26 acts against in this example is larger than that in prior examples, the driver 26 may include more than one spring device 30a/30b in order to provide a higher distributed force upon release.

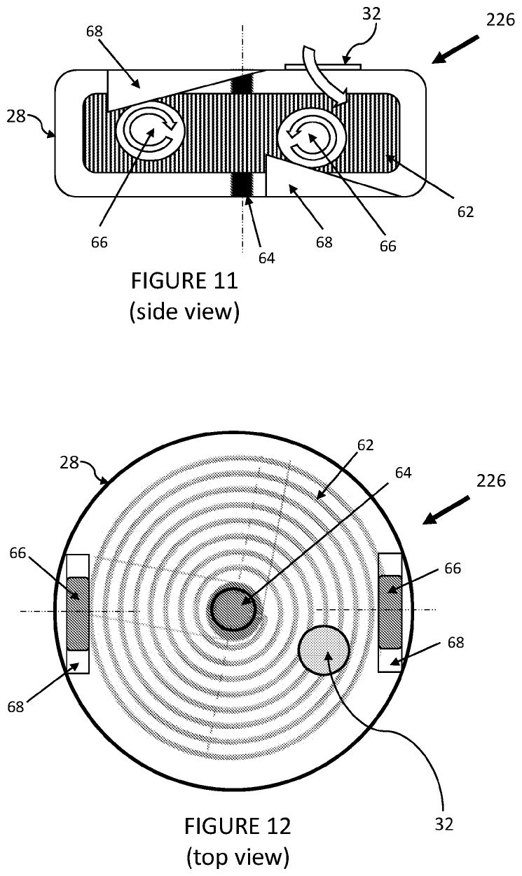

FIGS. 11 and 12 illustrate another example of a driver 226 that may be used in any of the prior example pumps. One contemplated use for the driver 226, however, is in a fluid bag, such as an intravenous (IV) fluid bag, for delivering IV solutions, hydration, blood, electrolytes, or nutrients to patients. The driver 226 includes a casing 28 and a constant force spring 62 in the casing 28 that is wound about an axle 64. A constant force spring (e.g., a clock spring) is a spring that is designed to provide a constant force over its range of motion. As an example, the constant force spring 62 includes narrow strip of spring steel that is wound into a spiral shape. When the spring is wound from its state of rest, it stores potential energy, and as the spring returns to it state of rest it exerts a constant force (which is usually coupled to one end of the spring, the other end being fixed).

The constant force spring 62 is coupled with a cam roller 66 (two shown) in the casing 28. The cam roller 66 is engaged with a cam track 68 in the casing 28. The casing 28 is evacuated to define a pressure differential that holds the constant force spring in a high energy state (e.g., wound) to provide the stored potential energy. Upon release of the pressure differential via the inlet port 32, the constant force spring 62 unwinds and rotates the cam roller 66 about the axle 64. The cam roller 66 is driven to ride on the cam track 68, which is in the shape of an inclined plane. Movement of the cam roller 66 against the cam track 68 drives the cam track 68 axially and thus expands the volume of the casing 28 to expand against the substance and draw in air through port 32.

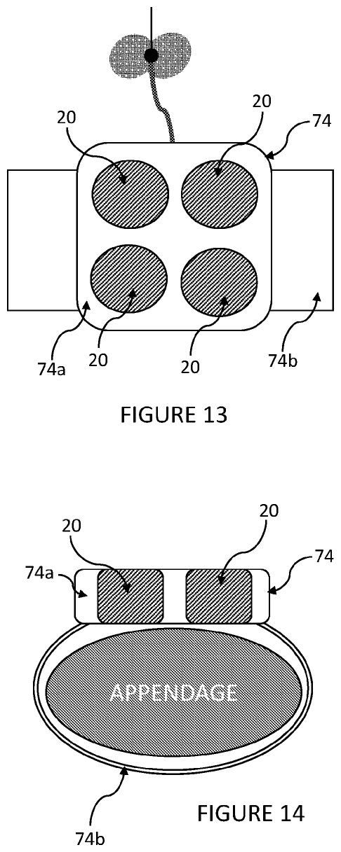

FIGS. 13 and 14 illustrate an example implementation in which there are multiple pumps 20 (120, 220, or 320) attached in a carrier 74. In this regard, the carrier 74 may include a housing 74a for holding the pumps 20 and an attachment member 74b that is configured to affix the pump housing 74a and pumps 20 to an object, such as the body of a patient. Thus, the patient can wear the pumps 20 during delivery of the substance. In this example, the attachment member 74b is an elastic band, but it is to be understood that other types of attachments could additionally or alternatively be used.

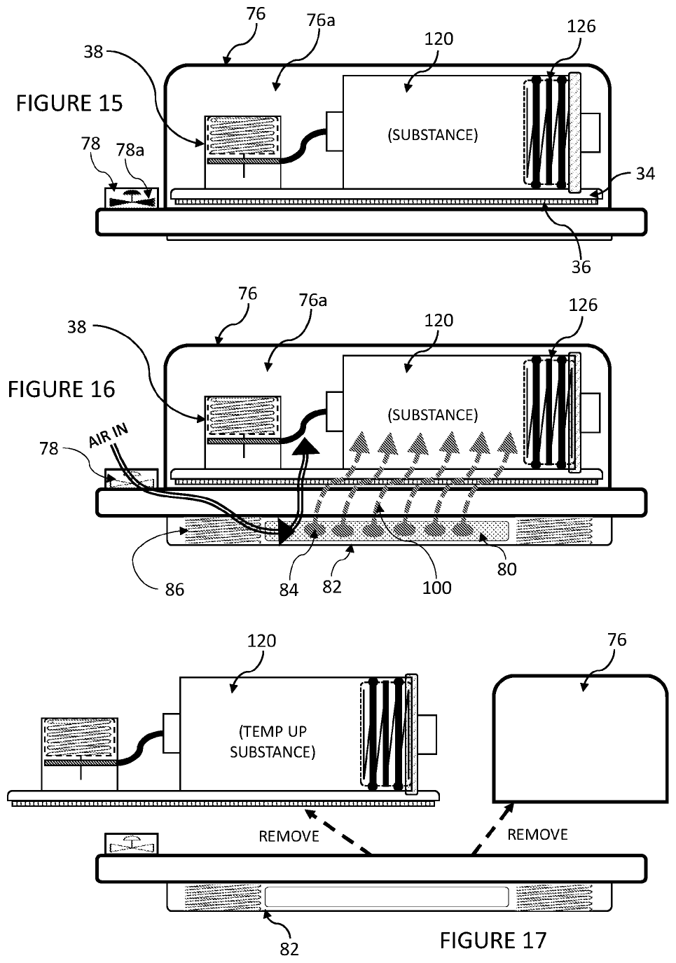

FIG. 15 illustrates another example of the pump 120, which in this case is sealed in a vessel 76 that has a pressurized chamber 76a. The pressure of the pressurized chamber 76a is greater than the evacuated pressure in the substance driver 126 in order to prevent premature release of the substance driver 126. For instance, particularly if shipping the pump 120, if the external pressure fluctuates and reaches a low enough level, the pressure differential may decrease to a point that the spring force of the spring device 30 is able to overcome the low pressure differential and prematurely release the driver 126. In this regard, the pressurized chamber 76a in the vessel maintains a constant pressure around the pump 126 to thereby ensure that the pressure differential across the driver 126 remains sufficiently high to prevent premature release. An inlet port 78 is connected with the pressurized chamber 76a for releasing the pressure in the vessel 76. For example, the inlet port 78 may include a valve 78a that may be opened to release the pressure when the pump 120 is to be removed from the vessel 76. The pressurized chamber 76a may be pressurized with air, but other gases may alternatively be used, such as gases that are inert with respect to the substance in the pump 20.

In a further example in FIGS. 16 and 17, there is additionally a heater module 80 attached with the vessel 67. The heater module 80 is operable to change the temperature of the substance in the pump 120 prior to removal of the pump 120 from the vessel 76 and/or, if attached to a patient, warm an area of the patient's body to increase blood flow and thus drug delivery. For example, the heater module 80 includes a heater module container 82 and a thermal material 84 in the heater module container 82. The thermal material 84 is exothermically reactive with air to produce heat. Initially, the heater module container 82 is evacuated. A heater module driver 86 is disposed in the heater module container 84, which is collapsible/expandable and functions like the casing 28 of the driver 26. The heater module driver 86 is constructed similar to the afore-mentioned drive 26 and has a stored potential energy that is releasable as kinetic energy. Upon release, the heater module driver 86 expands the heater module container 82 and thereby draws in air from at least one inlet 78 to provide air to the thermal material 84 and also to release the pressure in the pressure chamber 76a. Upon exposure to the air, the thermal material 84 produces heat that increases the temperature of the substance. The heat produced may additionally or alternatively be used to sterilize the inside of the vessel 76 and/or initiate a reaction in the substance(s) in the pump 20 to ready it for use.

The pumps disclosed herein may also be subject to an assembly process for fabrication. For example, a method for assembling a pump includes providing the container, introducing a medicament into the interior volume of the container, and then installing the substance driver at least partially into the interior volume of the container. The installing may involve providing the substance driver as a pre-fabricated and pre-evacuated component that is then attached to the container, such as by sliding the substance driver into an open end of the container. Alternatively, the substance driver may be attached to the container while the container is empty of a substance and then medicament substance is introduced through the exit port or a secondary fill port. Alternatively, the substance driver can be made in full or in part during the assembly. For instance, if the substance driver is not pre-evacuated, it may be evacuated to compress the spring device to its potential energy state prior to installation with the container, and this may occur before, after, or during the introduction of medicament to the container.

Although a combination of features is shown in the illustrated examples, not all of them need to be combined to realize the benefits of various embodiments of this disclosure. In other words, a system designed according to an embodiment of this disclosure will not necessarily include all of the features shown in any one of the Figures or all of the portions schematically shown in the Figures. Moreover, selected features of one example embodiment may be combined with selected features of other example embodiments.

The preceding description is exemplary rather than limiting in nature. Variations and modifications to the disclosed examples may become apparent to those skilled in the art that do not necessarily depart from this disclosure. The scope of legal protection given to this disclosure can only be determined by studying the following claims.

What is claimed is:

1. A pump comprising:
a container having an interior container volume for holding a substance;
an outlet associated with the interior container volume for discharging the substance;
a hollow needle connected with the outlet; and
a substance driver disposed in the interior container volume, the substance driver having a stored potential energy that is releasable as kinetic energy and, upon release, the substance driver expanding against the substance in the interior container volume and thereby discharging the substance through the outlet to the hollow needle.

2. The pump as recited in claim 1, wherein the outlet includes a conduit connecting the interior container volume with the hollow needle.

3. The pump as recited in claim 1, further comprising a needle container in which the hollow needle is disposed and a needle driver disposed in the needle container adjacent the hollow needle, the needle driver having a stored potential energy that is releasable as kinetic energy and, upon release, the needle driver expands and thereby drives the hollow needle to protrude from the needle container.

4. The pump as recited in claim 3, wherein release of the substance driver causes the substance to flow through the outlet to the hollow needle, and the pressure of the flowing substance moves an element to release the needle driver.

5. The pump as recited in claim 3, wherein the substance driver includes a casing and a spring device in the casing, the casing is evacuated to define a pressure differential that stresses the spring device to provide the stored potential energy, a pressure equalization port that is operable to release the pressure differential and, upon release, the spring device expands to increase the volume of the casing and thereby generate a vacuum in the casing.

6. The pump as recited in claim 5, wherein the pressure equalization port is connected by an inlet conduit to an inlet at the needle container, and the inlet is covered by a seal that is removeable to thereby release the pressure differential via the inlet conduit.

7. The pump as recited in claim 3, further comprising a needle cover on the needle container and a needle cover driver coupled with the needle cover, the needle cover driver having a stored potential energy that is releasable as kinetic energy and, upon release, the needle cover driver expanding and thereby moving the collapsible needle cover from store position to a deployed position shielding the hollow needle.

8. The pump as recited in claim 7, wherein the driving of the needle to protrude from the needle container causes the needle to pierce the needle cover driver and thereby release the needle cover driver.

9. The pump as recited in claim 8, wherein the driving of the needle to protrude from the needle container causes the needle to pierce the needle cover driver and release a flowable substance from inside the needle cover driver to the needle puncture site.

10. The pump as recited in claim 1, further comprising a base having opposed first and second sides, at least the container is attached on the first side, and the second side includes an adhesive.

11. The pump as recited in claim 1, wherein the container includes at least one flexible bladder with a fill port for holding the substance.

12. The pump as recited in claim 1, wherein the substance driver includes a casing and a constant force spring in the casing, the casing is evacuated to define a pressure differential that holds the constant force spring in a wound state around an axis to provide the stored potential energy, a pressure equalization port that is operable to release the pressure differential and, upon release, the constant force spring rotates to increase the volume of the casing to expand against the substance.

13. The pump as recited in claim 1, further comprising an attachment member configured to affix the pump to an object.

14. The pump as recited in claim 1, wherein the substance driver includes a casing and a spring device in the casing, the casing is evacuated to define a pressure differential that stresses the spring device to provide the stored potential energy, a pressure equalization port that is operable to release the pressure differential, and further comprising a vessel that has a pressurized chamber in which at least the container and the substance driver are disposed, the pressure of the pressurized chamber being greater than a pressure in the casing of the substance driver in order to prevent premature release of the substance driver.

15. The pump as recited in claim 14, further comprising a heater module attached with the vessel.

16. The pump as recited in claim 15, wherein the heater module includes a heater module container, a thermal material in the heater module container that is exothermically reactive with air to produce heat, a heater module driver disposed in the heater module container, the heater module driver having a stored potential energy that is releasable as kinetic energy and, upon release, the heater module driver expanding and drawing in air through at least one air inlet to the thermal material and produce heat.

17. A method for assembling a pump, the method comprising:
providing a container that has an interior volume and an outlet associated with the interior volume;
introducing a medicament into the interior volume;
installing a substance driver at least partially into the interior volume of the container, the substance driver has a stored potential energy that is releasable as kinetic energy and, upon release, the substance driver is expandable against the medicament in the interior volume to thereby discharge the substance through the outlet.

18. The method as recited in claim 17, further comprising evacuating an interior of the substance driver prior to installing the substance driver at least partially into the interior volume of the container.

19. A method of treatment comprising:
placing a pump on a patient, the pump has a container with an interior container volume that has a medicament, an outlet associated with the interior container volume to discharge the substance, and a substance driver disposed in the interior container volume, the substance driver has a stored potential energy that is releasable as kinetic energy;
connecting the outlet to the patient;
releasing the substance driver, the substance driver expanding against the medicament in the interior container volume and thereby discharging the medicament through the outlet and into the patient; and
after discharge of the medicament into the patient, removing the pump from the patient.

20. The method as recited in claim 19, wherein the connecting includes penetrating a hollow needle into the patient.
